(19) United States
(12) Patent Application Publication
(10) Pub. No.: US 2008/0147038 A1
(43) Pub. Date: Jun. 19, 2008

(54) LESION SITE MARKING DEVICE AND A METHOD OF MARKING A LESION SITE USING THE DEVICE

(76) Inventor: Grant T. Hoffman, Bloomington, IN (US)

Correspondence Address: BRINKS HOFER GILSON & LIONE/CHICAGO/COOK, PO BOX 10395, CHICAGO, IL, 60610, US

(21) Appl. No.: 11/959,839

(22) Filed: Dec. 19, 2007

Related U.S. Application Data
(60) Provisional application No. 60/875,852, filed on Dec. 19, 2006.

(51) Int. Cl. A61M 5/42

(52) U.S. Cl. 604/500; 604/116

(57) ABSTRACT

The invention is directed to lesion site marking devices and methods. Specifically, the device of this invention typically is made up of a filler body comprising a bioremodelable material comprising an extracellular matrix (ECM) material, such as small intestine submucosa (SIS) and a detectable marker associated with the filler body and adapted to mark a center of the filler body. The device of this invention may also be made up of a filler body comprising a biocompatible polymer, such as THORALON, and a detectable marker associated with the filler body and adapted to mark a center of the filler body.

### RELATED APPLICATIONS

The present patent document claims the benefit of the filing date under 35 U.S.C. §119(e) of Provisional U.S. Patent Application Ser. No. 60/875,852, filed Dec. 19, 2006, which is hereby incorporated by reference.

### BACKGROUND

1. Technical Field

This invention is directed to methods and marking devices including reconstituted or naturally derived extracellular matrix (ECM) material, such as small intestine submucosa (SIS) or biocompatible polymer materials.

2. Background Information

Tissue implants in a purified form and derived from collagen-based materials have been manufactured and disclosed in the literature. Cohesive films of high tensile strength have been manufactured using collagen molecules or collagen-based materials. Aldehydes, however, have been generally utilized to cross-link the collagen molecules to produce films having high tensile strengths. With these types of materials, the aldehydes may leech out of the film, e.g. upon hydrolysis. Because such residues are cytotoxic, the films are poor tissue implants.

Other techniques have been developed to produce collagen-based tissue implants while avoiding the problems associated with aldehyde crosslinked collagen molecules. One such technique is illustrated in U.S. Pat. No. 5,141,747, wherein the collagen molecules are cross-linked or coupled at their lysine epsilon amino groups followed by denaturing the coupled, and preferably modified, collagen molecules. The disclosed use of such collagen material is for tympanic membrane repair. While such membranes are disclosed to exhibit good physical properties and to be sterilized by subsequent processing, they are not capable of remodeling or generating cell growth or, in general, of promoting regrowth and healing of damaged or diseased tissue structures.

In general, researchers in the surgical arts have been working for many years to develop new techniques and materials for use as implants and grafts to replace or repair damaged or diseased tissue structures, for example, soft tissue, blood vessels, muscle, ligaments, tendons and the like.

It is not uncommon today, for instance, for a surgeon to use implantable prostheses formed from plastic, metal and/or ceramic material for reconstruction or replacement of physiological structures.

In the field of breast cancer, stereotactically guided and percutaneous biopsy procedures are common. However, following such a procedure, a patient is often left with a cavity from which a lesion was removed. Devices utilizing collagenous materials for filling a biopsy site have been previously suggested.

As mentioned above, various collagen-based materials have also been utilized for the above-mentioned tissue replacements; however, these materials either did not exhibit the requisite tensile strength or also had problems with infection and other immunogenic responses, encapsulation, or had other problems when they may have been loaded with antibiotics, growth factors and the like. For example, U.S. Pat. No. 4,956,178 discloses a submucosa collagen matrix which is obtained from the intestinal tract of mammals; however, it is disclosed that the collagen matrix is loaded with antibiotics. In a related patent, U.S. Pat. No. 5,372,821, it is disclosed that a submucosa collagen matrix may be sterilized by conventional techniques, e.g., aldehyde tanning, propylene oxide, gamma radiation and peracetic acid. No specific processing steps are disclosed except that the submucosa layer is first delaminated from the surrounding tissue prior to sterilization treatment. Also, even though gelatinous bioabsorbable materials, such as collagen, cross-linked collagen, and fibrin-collagen matrix have been suggested for filling a biopsy site in U.S. Pat. No. 6,356,782, no specific processing steps for the materials are disclosed.

Also, U.S. Pub. No. 2003/0206860 A1 discloses biomaterials that comprise a radiopaque collagenous material. Although these types of materials provide the advantage in that the radiopaque collagenous material is radiographic (easy to visualize) and soft, the use of only this material does not allow precise determination of the center or other location of a lesion that is being filled with the material.

Accordingly, improved devices and methods for visualization of a lesion site are desired.

### SUMMARY

In one embodiment, the invention is a lesion site marking device. The device includes a filler body comprising an expandable bioremodelable material comprising an ECM material and a detectable marker attached to the filler body and adapted to mark a center of the filler body. Preferably the ECM material includes submucosa, renal capsule membrane, dermal collagen, dura mater, pericardium, fascia lata, serosa, peritoneum or basement membrane layers, including liver basement membrane, intestinal submucosa, small intestinal submucosa, stomach submucosa, urinary bladder submucosa, and uterine submucosa. The bioremodelable material preferably includes SIS. Preferably, SIS is fluidized. SIS may be comminuted. Preferably, the marker is mammographic. The marker may also be radiopaque and/or echogenic. Preferably, the marker is located within an interior of the filler body.

In another embodiment, the invention is a lesion site marking device that includes a filler body comprising an expandable biocompatible polymer comprising a polyurethane urea and a detectable marker attached to the filler body and adapted to mark a center of the filler body. Preferably, the polymer comprises a polyetherurethane urea blended with a siloxane containing surface modifying additive. More preferably, polymer comprises a base polymer and about 0.5% to about 5% by weight of the base polymer of a surface modifying additive, wherein the surface modifying additive comprises polydimethylsiloxane and the reaction product of diphenylmethane diisocyanate and 1,4-butanediol, and wherein the base polymer is a polyetherurethane urea comprising polytetramethylene oxide and the reaction product of 4,4′-diphenylmethane diisocyanate and ethylene diamine.

In another embodiment, the invention is a method of marking a tissue lesion site having a margin in a mammalian body. The method includes the steps of subcutaneously accessing the lesion site via a delivery device and deploying a lesion site marking device comprising expandable bioremodelable material comprising an ECM material and a detectable marker attached to the filler body and adapted to mark a center of the filler body. Upon delivery into the lesion site, the lesion site marking device assumes a pre-determined three-dimensional configuration so to (a) substantially fill the lesion site, (b) mark the lesion site margin, and (c) indicate the orientation of the marker inside the lesion site. Preferably, the delivery device includes a biopsy device.

### BRIEF DESCRIPTION OF THE DRAWINGS

FIG. 1 illustrates an exemplary lesion site marking device with a filler body and a single, centrally located marker; and

FIG. 2 illustrates another exemplary lesion site marking device with a filler body having an outer covering.

### DETAILED DESCRIPTION OF THE DRAWINGS AND THE PRESENTLY PREFERRED EMBODIMENTS

For the purpose of promoting an understanding of the principles of the invention, reference will now be made to certain preferred embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

In the discussions herein, a number of terms are used. In order to provide and clear and consistent understanding of the specification and claims, the following definitions are provided.

“Bioburden” refers to the number of living microorganisms, reported in colony-forming units (CFU), found on and/or in a given amount of material. Illustrative microorganisms include bacteria, fungi and their spores.

“Disinfection” refers to a reduction in the bioburden of a material.

“Sterile” refers to a condition wherein a material has a bioburden such that the probability of having one living microorganism (CFU) on and/or in a given section of the material is one in one-million or less.

“Pyrogen” refers to a substance which produces febrile response after introduction into a host.

“Endotoxin” refers to a particular pyrogen which is part of the cell wall of gram-negative bacteria. Endotoxins are continually shed from the bacteria and contaminate materials.

“Purification” refers to the treatment of a material to remove one or more contaminants which occur with the material, for instance contaminants with which the material occurs in nature, and/or microorganisms or components thereof occurring on the material. Illustratively, the contaminants may be those known to cause toxicity, infectivity, pyrogenicity, irritation potential, reactivity, hemolytic activity, carcinogenicity and/or immunogenicity.

“Biocompatibility” refers to the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests assay as to a material's toxicity, infectivity, pyrogenicity, irritation potential, reactivity, hemolytic activity, carcinogenicity and/or immunogenicity. A biocompatible structure or material when introduced into a majority of patients will not cause an adverse reaction or response. In addition, it is contemplated that biocompatibility can be effected by other contaminants, such as prions, surfactants, oligonucleotides, and other biocompatibility effecting agents or contaminants.

“Contaminant” refers to an unwanted substance on, attached to, or within a material. This includes, but is not limited to: bioburden, endotoxins, processing agents such as antimicrobial agents, blood, blood components, viruses, DNA, RNA, spores, fragments of unwanted tissue layers, cellular debris, and mucosa.

“Tela submucosa” refers to a layer of collagen-containing connective tissue occurring under the mucosa in most parts of the alimentary, respiratory, urinary and genital tracts of animals.

As disclosed above, the present invention generally provides procedures and devices for marking a biopsy site (i.e., lesion site). Advantageous devices of the invention are made up of a bioremodelable material, such as reconstituted or naturally derived collagenous material, including SIS, which forms a filler body. The devices of this invention also include at least one preferably radiopaque or echogenic marker associated with the filler body, which in certain embodiments marks the center of the filler body.

As mentioned above, the filler body is made up of a remodelable, and particularly a remodelable collagenous material. Such remodelable collagenous material can be provided, for example, by collagenous materials isolated from suitable tissue source from a warm-blooded vertebrate, and especially a mammal. Such isolated collagenous materials may be processed so as to have remodelable properties and promote cellular invasion and tissue infiltration. Remodelable materials may be used in this context to promote cellular growth or ingrowth at places of contact with tissue, while optionally containing others materials.

Reconstituted or naturally-derived collagenous materials may be used to form the filler body of the device of the present invention. Such materials that are at least bioresorbable will provide advantage in the present invention, with materials that are bioremodelable and promote cellular invasion and ingrowth providing particular advantage.

Suitable bioremodelable materials may be provided by collagenous extracellular matrix materials (ECMs) possessing biotropic properties, including in certain forms angiogenic collagenous ECMs. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, protein, or gene expression.

Submucosa or other ECM materials may be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances.

Illustrative drug substances that may be incorporated into and/or onto the ECM materials used in the invention include, for example agents that inhibit bacterial or microbial activity. Suitable anti-infective agents include: anthracyclines (e.g., doxorubicin and mitoxantrone), fluoropyrimidines, folic acid antagonists (e.g., methotrexate), podophylotoxins (e.g., etoposide), camptothecins, and hydroxyureas. Particular nonlimiting examples of antimicrobial agents that may be used include acyclovir, amantadine, aminoglycosides (e.g., amikacin, gentamicin and tobramycin), amoxicillin, amoxicillin/Clavulanate, amphotericin B, ampicillin, ampicillin/sulbactam, atovaquone, azithromycin, cefazolin, cefepime, cefotaxime, cefotetan, cefpodoxime, ceftazidime, ceftizoxime, ceftriaxone, cefuroxime, cefuroxime axetil, cephalexin, chloramphenicol, clotrimazole, ciprofloxacin, clarithromycin, clindamycin, dapsone, dicloxacillin, doxycycline, erythromycin, fluconazole, foscamet, ganciclovir, atifloxacin, imipenem/cilastatin, isoniazid, itraconazole, ketoconazole, metronidazole, nafcillin, nafcillin, nystatin, penicillin, penicillin G, pentamidine, piperacillin/tazobactam, rifampin, quinupristin-dalfopristin, ticarcillin/clavulanate, trimethoprim/sulfamethoxazole, valacyclovir, vancomycin, mafenide, silver sulfadiazine, mupirocin, nystatin, triamcinolone/nystatin, clotrimazole/betamethasone, clotrimazole, ketoconazole, butoconazole, miconazole, tioconazole, detergent-like chemicals that disrupt or disable microbes (e.g., nonoxynol-9, octoxynol-9, benzalkonium chloride, menfegol, and N-docasanol); chemicals that block microbial attachment to target cells and/or inhibits entry of infectious pathogens (e.g., sulphated and sulponated polymers such as PC-515 (carrageenan), Pro-2000, and Dextrin 2 Sulphate), antiretroviral agents (e.g., PMPA gel) that prevent retroviruses from replicating in the cells; genetically engineered or naturally occurring antibodies that combat pathogens such as anti-viral antibodies genetically engineered from plants known as “plantibodies;” agents which change the condition of the tissue to make it hostile to the pathogen (such as substances which alter mucosal pH (e.g., Buffer Gel and Acidform) or non-pathogenic or “friendly” bacteria or other microbes that cause the production of hydrogen peroxide or other substances that kill or inhibit the growth of pathogenic microbes (e.g., lactobacillus).

Illustrative drug substances that may be incorporated into and/or onto the ECM materials used in the invention include, for example agents that reduce or alleviate pain, including analgesic, antipyretic, and non-steroidal antiinflammatory agents (NSAIDs). Specifically, these agents may include salicylic acid derivatives (e.g., Asprin, sodium salicylate, choline magnesium trisalicylate, salsalate, diflunisal, salicylsalicylic acid, sulfasalazine, olsalazine), para-aminophenol derivatives (e.g., Acetaminophen), indole and indene acetic acids (e.g., indomethacin, sulindac, etodolac), heteroaryl acetic acids (e.g., Tolmetin, diclofenac, ketorolac), arylpropionic acids (Ibuprofen, naproxen, flurbiprofen, ketoprofen, fenoprofen, oxaprozin), anthranilic acids (fenamates, such as mefenamic acid, meclofenamic acid), enolic acids (oxicams, such as piroxicam and tenoxicam, pyrazolidinediones, such as phenylbutazone and oxyphenthatrazone), and alkanones (e.g., nabumetone). Additional agents include, for example, selective COX-1 and COX-2 inhibitors (Bextra, Celebrex, and Vioxx). Further agents include, for example, opioids (agonists/antagonists), such as codeine, oxycodone and morphine, and other morphine-like drugs, such as methadone. Opioids may also be combined with small doses of amphetamine to augment analgesia while reducing the sedative effects.

Illustrative drug substances that may be incorporated into and/or onto the ECM materials used in the invention also include, for example agents that treat or prevent an allergic or immune response, such as cytokine inhibitors (including humanized anti-cytokine antibodies, anti-cytokine receptor antibodies), recombinant (new cell resulting from genetic recombination) antagonists, or soluble receptors), various leucotriene modifiers (including zafirlukast, montelukast and zileuton), immunoglobulin E (IgE) inhibitors (including Omalizumab and secretory leukocyte protease inhibitors). Suitable immunomodulatory agent, such as sirolimus or rapamycin analogues and derivatives including tacrolimus, everolimus and ABT-578 (zotarolimus) may also be used.

Illustrative drug substances that may be incorporated into and/or onto the ECM materials used in the invention also include, for example agents that treat a tumor or cancerous lesion, such as antitumor agents (e.g., cancer chemotherapeutic agents, biological response modifiers, vascularization inhibitors, hormone receptor blockers, cryotherapeutic agents or other agents that destroy or inhibit neoplasia or tumorigenesis), alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase inhibitors, such as aminoglutethamide and formestane, trriazole inhibitors, such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000), which is expressly incorporated herein by reference, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxanes (e.g., paclitaxel and derivatives thereof including docetaxel), and taxotere.

Illustrative drug substances that may be incorporated into and/or onto the ECM materials used in the invention also include, for example agents that may be selected to inhibit fibrosis. Examples of suitable fibrosis-inhibiting agents include an antimycotic agent, such as miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole, piperazine, isoconazole, griseofulvin, bifonazole, econazole nitrate, croconazole, sertaconazole, omoconazole, flutrimazole, fluconazole, neticonazole, monohydrochloride, butoconazole, clotrimazole, or a collagen antagonist, such as benzenepropanamide, lufironil, or an analogue or derivative thereof.

In certain embodiments, thrombus-promoting substances, such as blood clotting factors, e.g., thrombin, fibrinogen, and the like, as well as hemostatic agents may also be incorporated.

In other embodiments, incorporating other healing and therapeutic substances is also contemplated.

These substances may be applied to the ECM material as a premanufactured step, immediately prior to the procedure (e.g., by soaking the material in a solution containing a suitable antibiotic such as cefazolin), or during or after delivery of the material in the patient.

Submucosa or other ECM tissue used in the invention is preferably highly purified, for example, as described in U.S. Pat. No. 6,206,931, which is incorporated by reference herein. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

Preferred type of submucosa for use in this invention is derived from the intestines, more preferably the small intestine, of a warm blooded vertebrate; i.e., SIS. SIS is commercially available from Cook Biotech, West Lafayette, Ind.

Preferred intestine submucosal tissue typically includes the tunica submucosa delaminated from both the tunica muscularis and at least the luminal portions of the tunica mucosa. In one example the submucosal tissue includes the tunica submucosa and basilar portions of the tunica mucosa including the lamina muscularis mucosa and the stratum compactum. The preparation of intestinal submucosa was described in U.S. Pat. No. 4,902,508, and the preparation of tela submucosa was described in U.S. Pat. No. 6,206,931, both of which are incorporated herein by reference. The preparation of submucosa was also described in U.S. Pat. No. 5,733,337 and in 17 Nature Biotechnology 1083 (November 1999); and WIPO Publication WO 98/22158, which is the published application of PCT/US97/14855. Also, a method for obtaining a highly pure, delaminated submucosa collagen matrix in a substantially sterile state was previously described in U.S. Publication No. 2004/0180042 A1, disclosure of which is incorporated by reference.

The submucosal tissue may be processed to provide fluidized compositions, for instance using techniques as described in U.S. Pat. No. 5,275,826. In this regard, solutions or suspensions of submucosa can be prepared by comminuting and/or digesting the submucosa with a protease (e.g. trypsin or pepsin), for a period of time sufficient to solubilize the tissue and form substantially homogeneous solution. The submucosa starting material is desirably comminuted by tearing, cutting, grinding, shearing or the like. Grinding the submucosa in a frozen or freeze-dried state is advantageous, although good results can be obtained as well by subjecting a suspension of pieces of the submucosa to treatment in a high speed blender and dewatering, if necessary, by centrifuging and decanting excess waste. The comminuted submucosa can be dried, for example freeze dried, to form a powder. Thereafter, if desired, the powder can be hydrated, that is, combined with water or buffered saline and optionally other pharmaceutically acceptable excipients, to form a fluid composition, e.g. having a viscosity of about 2 to about 300,000 cps at 25EC. The higher viscosity compositions can have a gel or paste consistency.

In one illustrative preparation, submucosa prepared as described herein may be reduced to small pieces (e.g. by cutting) which are charged to a flat bottom stainless steel container. Liquid nitrogen is introduced into the container to freeze the specimens, which are then comminuted while in the frozen state to form a coarse submucosa powder. Such processing can be carried out, for example, with a manual arbor press with a cylindrical brass ingot placed on top of the frozen specimens. The ingot serves as an interface between the specimens and the arbor of the press. Liquid nitrogen can be added periodically to the submucosa specimens to keep them frozen.

Other methods for comminuting submucosa specimens can be utilized to produce a submucosa powder. For example, submucosa specimens can be freeze-dried and then ground using a manual arbor press or other grinding means. Alternatively, submucosa can be processed in a high shear blender to produce, upon dewatering and drying, a submucosa powder.

Further grinding of the submucosa powder using a prechilled mortar and pestle can be used to produce consistent, more finely divided product. Again, liquid nitrogen is used as needed to maintain solid frozen particles during final grinding. The powder can be easily hydrated using, for example, buffered saline to produce a fluidized composition for use in this invention at the desired viscosity.

To prepare another preferred fluidized material, a submucosa powder can be sifted through a wire mesh, collected, and subjected to proteolytic digestion to form a substantially homogeneous solution. For example, the powder can be digested with 1 mg/ml of pepsin (Sigma Chemical Co., St. Louis Mo.) and 0.1 M acetic acid, adjusted to pH 2.5 with HCl, over a 48 hour period at room temperature. After this treatment, the reaction medium can be neutralized with sodium hydroxide to inactivate the peptic activity. The solubilized submucosa can then be concentrated by salt precipitation of the solution and separated for further purification and/or freeze drying to form a protease-solubilized intestinal submucosa in powder form.

It is also possible to form large surface area constructs by combining two or more submucosa segments, for instance using techniques as described in U.S. Pat. No. 2,127,903 and/or International Publication No. WO 96/32146, dated 17 Oct. 1996, publishing International Application No. PCT/US96/04271, filed 5 Apr. 1996. Thus, a plurality of submucosa strips can be fused to one another, for example by compressing overlapping areas of the strips under dehydrating conditions, to form an overall planar construct having a surface area greater than that of any one planar surface of the individual strips used to form the construct.

Comminuted forms of submucosa and methods of obtaining same were previously described in U.S. Pat. Nos. 5,275,826 and 5,516,533, which are incorporated herein by reference.

The ECM material, such as SIS for use in the present invention may be processed to provide preferred shape or form of the ECM material. For example, the ECM material may take many shapes and forms, such as string or fiber-like, filament, thread, coiled; helical; spring-like; randomized; branched; sheet-like; tubular; spherical; fragmented; powdered; ground; sheared; fluidized; gel-like; sponge-like; foam-like; and solid material sheet-like. Sponge-like and foam-like forms of the ECM materials are preferred for use in this invention. In certain embodiments, fluidized or gel-like forms of the ECM material may be preferred.

Solid and fluidized bioremodelable materials, such as submucosa compositions find wide application in tissue replacement, augmentation, and/or repair. Specifically, the submucosal compositions, such as SIS, can be used to induce regrowth of natural tissue in an area of biopsy lesion resulting from stereotactically guided and percutaneous biopsy procedures of breast tissue. By delivering an effective amount of a submucosa composition into the locale of a biopsy lesion in need of healing, one can readily take advantage of the biotropic properties of the submucosa.

Specifically, solid and fluidized submucosa compositions can be used to form a filler body, and in combination with a marker, can be used to form a lesion site marking device of this invention.

For example, SIS can be used as a cell growth substrate, illustratively in sheet, paste or gel form in combination with nutrients which support the growth of the subject cells, e.g. eukaryotic cells (see, e.g. International Publication No. WO 96/24661 dated 15 Aug. 1996, publishing International Application No. PCT/US96/01842 filed 9 Feb. 1996) and to form a filler body of the lesion site marking device. As mentioned above, SIS can be used alone, or in combination with one or more additional bioactive agents such as physiologically compatible minerals, growth factors, antibiotics, chemotherapeutic agents, antigen, antibodies, enzymes and hormones. In preferred forms, the SIS composition will support the proliferation and/or differentiation of mammalian cells, including human cells.

While naturally derived biomaterials, particularly bioremodelable materials like SIS described above, are generally preferred for use in this invention, synthetic materials, including those into which growth factors are added to make them bioremodelable, are also within the scope of this invention. One example of suitable material that may be used in this invention is biocompatible porous THORALON (THORATEC, Pleasanton, Calif.) as described in U.S. Pat. Nos. 4,675,361 and 6,939,377, both of which are incorporated herein by reference. Preferably, THORALON is in a form of foam.

THORALON is a polyurethane base polymer blended (referred to as BPS-215) with a siloxane containing surface modifying additive (referred to as SMA-300). The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. Porous THORALON can be formed by mixing the polyetherurethane urea (BPS-215), the surface modifying additive (SMA-300) and a particulate substance in a solvent. The particulate may be any of a variety of different particulates, pore forming agents or inorganic salts. Preferably the particulate is insoluble in the solvent. Examples of solvents include dimethyl formamide (DMF), tetrahydrofuran (THF), dimethyacetamide (DMAC), dimethyl sulfoxide (DMSO), or mixtures thereof. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water f. or the extraction, for example water at a temperature of about 60° C. The resulting pore diameter can be substantially equal to the diameter of the salt grains.

The porous polymeric sheet can have a void-to-volume ratio from about 0.40 to about 0.90. Preferably the void-to-volume ratio is from about 0.65 to about 0.80. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter is measured based on images from a scanning electron microscope (SEM). Formation of porous THORALON is described, for example, in U.S. Pat. No. 6,752,826 and U.S. Patent Application Publication No. 2003/0149471 A1, both of which are incorporated herein by reference.

Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes may also be employed. These include polyurethane ureas that preferably include a soft segment and include a hard segment formed from a diisocyanate and diamine. For example, polyurethane ureas with soft segments such as polytetramethylene oxide, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

The diisocyanate used as a component of the hard segment may be represented by the formula OCN—R—NCO, where —R— may be aliphatic, aromatic, cycloaliphatic or a mixture of aliphatic and aromatic moieties. Examples of diisocyanates include tetramethylene diisocyanate, hexamethylene diisocyanate, trimethyhexamethylene diisocyanate, tetramethylxylylene diisocyanate, 4,4′-decyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, neopentyl alcohol, 1,6-hexanediol, 1,8-octanediol, propylene glycols, 2,3-butylene glycol, dipropylene glycol, dibutylene glycol, glycerol, or mixtures thereof.

Biocompatible polyurethanes modified with cationic, anionic and aliphatic side chains may also be used. See, for example, U.S. Pat. No. 5,017,664.

Other biocompatible polyurethanes include: segmented polyurethanes, such as BIOSPAN; polycarbonate urethanes, such as BIONATE; and polyetherurethanes such as ELASTHANE; (all available from POLYMER TECHNOLOGY GROUP, Berkeley, Calif.).

Other biocompatible polyurethanes include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible polyurethanes may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

Examples of other biocompatible polyurethanes include BIOSPAN, BIONATE, ELASTHANE, PURSIL and CARBOSIL (POLYMER TECHNOLOGY GROUP, Berkeley, Calif.).

Drug substances may also be incorporated into and/or onto the biocompatible polymer materials used in the invention and include those described above in connection with the ECM.

A lesion site marking device of this invention may have various suitable configurations. Exemplary configurations include spherical, cylindrical, conical, ellipsoidal, or a multi-faced or irregular etc. filler body. Other shapes are also contemplated. For example, the filler body may also have an irregular or random shape, in the case of a gel, combining features of various curved and planar surfaces. Preferably, the filler body can assume a variety of shapes and be expandable. For example, the filler body may be constructed to have substantially curved surfaces, such as the preferred spherical and cylindrical. Preferably, the particular filler body shape will be chosen to best match to the biopsy or lesion site in which the device is placed. In one embodiment, the filler body shape can be chosen to be considerably larger than the lesion site.

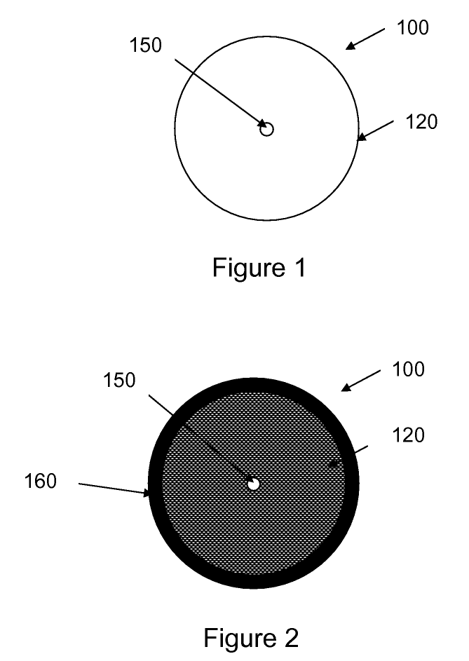

As illustrated in FIG. 1, together, a marker 150 and submucosa, which forms a filler body 120, are used to form a lesion site marking device 100 of this invention.

In certain embodiments, the marker may be located at or near the geometric center of the filler body. Alternatively, the marker 150 may reside in a location other than in center of the filler body. It is, however, preferred that marker 150 is located in a predetermined, preferably central, location and orientation in the device body so to aid the physician in determining the location and orientation of the biopsy site. Various forms of possible markers and exemplary placement locations for the marker in the filler body were previously illustrated in U.S. Pat. No. 6,356,782.

Preferably, the marker is radiopaque, echogenic, mammographic (“mammographic” means that the component described is visible under radiography or any other traditional or advanced mammography technique in which breast tissue is imaged), etc. so that it can be located by non-invasive techniques. This feature may be an inherent property of the material used for the marker. For radiopacity, the marker may be made of a non-bioabsorbable radiopaque material such as platinum, platinum-iridium, platinum-nickel, platinum-tungsten, gold, silver, rhodium, tungsten, tantalum, titanium, nickel, nickel-titanium, their alloys, and stainless steel or any combination of these metals.

A coating or the like may also be added to the marker to render the marker detectable or to enhance its detectability. The coating may be a substance that allows for the non-invasive detection of the marker. For radiopaque coating, elements such as barium- and bismuth-containing compounds, as well as particulate radio-opaque fillers, e.g., powdered tantalum or tungsten, barium carbonate, bismuth oxide, barium sulfate, etc. are preferred. To aid in detection by ultrasound or similar imaging techniques, any component of the device may be combined with an echogenic coating (e.g., ECHO-COAT from STS Biopolymers).

The marker may be affixed or attached to the interior or on the surface of the filler body by any number of suitable methods. For instance, the marker may be suspended in the interior of the body (especially in the instance where the filler body is a gel, such as fluidized SIS), it may be woven into the body (especially in the case where the marker is a wire or suture and the filler body is a solid SIS), it may be press fit onto the filler body (especially in the case where the marker is a ring or band), it may affixed to the filler body by a biocompatible adhesive, or it may be incorporated into the filler body during the process of forming the filler body. Any suitable means to affix, attach or suspend the marker into the filler body in the preferred location is within the scope of the present invention and will be known to a skilled artisan.

In additional embodiment, shown in FIG. 2, the filler body 120 may be enveloped in at least one outer covering 160 consisting of a layer of bioabsorbable material. Examples of suitable synthetic bioabsorbable polymers that may be used for the outer covering of the device are polyglycolide, or polyglycolic acid (PGA), polylactide, or polylactic acid (PLA), poly ε-caprolactone, polydioxanone, polylactide-co-glycolide, e.g., block or random copolymers of PGA and PLA, and other commercial bioabsorbable medical polymers. Such a covering can be radiopaque and/or echogenic, or it may be augmented with an additional coating of an echogenic and/or radiopaque material. The covering can also be made to be palpable so that the physician or patient can be further aided in determining the location and integrity of the implanted inventive device.

The covering may be designed to have a varying bioabsorption rate depending upon the thickness and type of material making up the covering. Preferably, the covering can be designed to degrade over a period of time ranging from as long as a year or more to as little as several months, weeks, or even days. It is preferred that such a bioabsorbable covering be designed to degrade between one and six months; especially preferred is three months. Other time periods are also contemplated depending on the type and thickness and number of layers of the material making up the covering.

As shown in FIG. 2, interior of the filler body 120 is SIS that is readily bioremodeled by the human or mammalian body once the covering 160 degrades. Interior may be filled with solid or gelatinous SIS material that can be optionally made radiopaque by any number of techniques known to one of skilled in the art.

The lesion site marking device of this invention may be delivered into the body either surgically via an opening in the body cavity, or through a minimally invasive procedure using such devices as a catheter, introducer, biopsy device or similar type device. Upon insertion of the lesion site marking device into the biopsy site, the lesion site marking device can self-expand and substantially fill the lesion site.

Exemplary methods that may be employed to place a device in the biopsy site were previously described in U.S. Pat. No. 6,356,782, which is incorporated herein by reference in its entirety, and will be known to one skilled in the art.

For example, the lesion site marking device may be delivered percutaneously through the same access device used to perform the biopsy in which tissue was removed from lesion site. The delivery preferably occurs immediately after removal of the biopsy. Although this is not necessary, it is less traumatic to the patient and allows more precise placement of the lesion site marking device before fluid begins to fill the biopsy site. Exemplary access devices were previously described in U.S. Pat. Nos. 6,136,014 and 6,036,698, the entirety of each are which hereby incorporated by reference. Any suitable access device may be used and will be know to one skilled in the art. For instance, a suitable tubular percutaneous access device, such as a catheter or delivery tube may be used to deliver the lesion site marking device of the present invention. Alternatively, the lesion site marking device of the present invention may be delivered to the lesion site by a plunger that is capable of both advancing the lesion site marking device and delivering a bio-compatible fluid, such as a saline solution or water, which may aid with the expansion of the filler body. In certain embodiments, the lesion site marking device may be delivered to the lesion site by using a bio-compatible fluid as the force to deliver the marking device into the lesion site.

In one embodiment, the invention further includes filling the biopsy site with a bioremodelable liquid, fluidized or gel-like material, preferably fluidized ECM, such as SIS, allowing the material to partially solidify or gel and then placing a marker, which may have a configuration as described above, into the center or near the center of the bioremodelable SIS material. The fluidized ECM may also be made radiopaque or echogenic by the addition of radiopaque materials, such as barium- or bismuth-containing compounds and the like, as well as particulate radio-opaque fillers, e.g., powdered tantalum or tungsten, barium carbonate, bismuth oxide, barium sulfate, to the gel.

Once the lesion site marking device is delivered to the lesion site, a follow-up noninvasive detection techniques, such as x-ray mammography or ultrasound may be used by a physician to identify, locate, and monitor the biopsy site over a preferred period of time.

After placement of the lesion site marking device into the lesion site, the bioremodelable SIS filler body promotes cell ingrowth and bioremodeling at a predetermined rate. As the filler body of the lesion site marking device is remodeled, the marker, which is usually suspended substantially in the volumetric center of the body of the device, is left in the center of the lesion site. This allows the physician to determine the location as well as the periphery of the biopsy site during subsequent examinations.

As previously mentioned, both the filler body and the marker are preferably made, via radiopaque or echogenic coatings, to bioremodel and/or degrade and/or absorb into the patient's body over a predetermined period of time. It is generally preferred that if the marker's radiopacity or echogenicity is chosen to degrade over time, such degradation does not take place within at least one year after implantation of the inventive device. In this way, if a new lump or calcification (in the case of a breast biopsy) is discovered after the biopsy, such a marker will allow the physician to know the relation of such new growth in relation to the region of excised tissue.

In another embodiment of this invention that either or both of the marker or markers and the bioremodelable body may be radioactive, if a regimen of treatment using radioactivity is contemplated.

From the foregoing, it is understood that the invention provides an improved lesion site marking device and method. While the above descriptions have described the invention for use in the marking of biopsy sites; i.e. lesion sites, the invention is not limited to such. One such application is evident as the invention may further be used as a lumpectomy site marker. In this use, the lesion site marking device may yield an improved benefit by marking the perimeter of the lumpectomy cavity or site. Also, the device of this invention may be used in any internal, preferably soft, tissue, but is most useful in breast tissue, lung tissue, prostate tissue, lymph gland tissue, etc.

It is therefore intended that the foregoing detailed description be regarded as illustrative rather than limiting, and that it be understood that it is the following claims, including all equivalents, that are intended to define the spirit and scope of this invention.

1. A lesion site marking device comprising
a filler body comprising an expandable bioremodelable material comprising an extracellular matrix material;
a detectable marker attached to the filler body and adapted to mark a center of the filler body.

2. The lesion site marking device of claim 1, wherein the extracellular matrix material is selected from the group consisting of submucosa, renal capsule membrane, dermal collagen, dura mater, pericardium, fascia lata, serosa, peritoneum or basement membrane layers, including liver basement membrane, intestinal submucosa, small intestinal submucosa, stomach submucosa, urinary bladder submucosa, and uterine submucosa.

3. The lesion site marking device of claim 2, wherein the extracellular matrix material comprises a radiopaque additive.

4. The lesion site marking device of claim 1, wherein the bioremodelable material comprises small intestine submucosa.

5. The lesion site marking device of claim 4, wherein the small intestine submucosa is fluidized.

6. The lesion site marking device of claim 4, wherein the small intestine submucosa is comminuted.

7. The lesion site marking device of claim 1, wherein the marker comprises material selected from the group consisting of platinum, iridium, nickel, tungsten, tantalum, gold, silver, rhodium, titanium, alloys thereof, and stainless steel.

8. The lesion site marking of claim 7, wherein the marker further comprises a polymer having a radiopaque additive.

9. The lesion site marking device of claim 8, wherein the radiopaque additive is selected from the group consisting of barium-containing compounds, bismuth-containing compounds, powdered tantalum, powdered tungsten, barium carbonate, bismuth oxide, and barium sulfate.

10. The lesion site marking device of claim 1, wherein the marker is mammographic, radiopaque, or echogenic.

11. The lesion site marking device of claim 1, wherein the filler body is radiopaque.

12. The lesion site marking device of claim 1, wherein the marker is located within an interior of the filler body.

13. A lesion site marking device comprising
a filler body comprising an expandable biocompatible polymer comprising a polyurethane urea;
a detectable marker attached to the filler body and adapted to mark a center of the filler body.

14. The lesion site marking device of claim 13, wherein the polymer comprises a polyetherurethane urea blended with a siloxane containing surface modifying additive.

15. The lesion site marking device of claim 13, wherein the polymer comprises a base polymer and about 0.5% to about 5% by weight of the base polymer of a surface modifying additive,
wherein the surface modifying additive comprises polydimethylsiloxane and the reaction product of diphenylmethane diisocyanate and 1,4-butanediol; and
wherein the base polymer is a polyetherurethane urea comprising polytetramethylene oxide and the reaction product of 4,4′-diphenylmethane diisocyanate and ethylene diamine.

16. A method of marking a tissue lesion site having a margin in a mammalian body, comprising:
subcutaneously accessing the lesion site via a delivery device; and
deploying a lesion site marking device comprising expandable bioremodelable material comprising an extracellular matrix material and a detectable marker attached to the filler body and adapted to mark a center of the filler body;
wherein upon delivery into the lesion site the lesion site marking device assumes a pre-determined three-dimensional configuration so to (a) substantially fill the lesion site, (b) mark the lesion site margin, and (c) indicate the orientation of the marker inside the lesion site.

17. The method of claim 16, wherein the extracellular matrix material is selected from the group consisting of submucosa, renal capsule membrane, dermal collagen, dura mater, pericardium, fascia lata, serosa, peritoneum or basement membrane layers, including liver basement membrane, intestinal submucosa, small intestinal submucosa, stomach submucosa, urinary bladder submucosa, and uterine submucosa.

18. The method of claim 16, wherein the bioremodelable material comprises small intestine submucosa.

19. The method of claim 18, wherein small intestine submucosa is fluidized.

20. The method of claim 16, wherein the delivery device comprises a biopsy device.
